(19) United States
(12) Patent Application Publication
(10) Pub. No.: US 2020/0291017 A1
(43) Pub. Date: Sep. 17, 2020

(54) CITRATE SALT OF THE COMPOUND (S)-4-((S)-3-FLUORO-3-(2-(5,6,7,8-TETRAHYDRO-1,8-NAPHTHYDRIN-2-YL)ETHYL)PYRROLIDIN-1-YL)-3-(3-(2-METHOXYETHOXY)PHENYL) BUTANOIC ACID

(71) Applicant: GlaxoSmithKline Intellectual Property Development Limited, Brentford, Middlesex (GB)

(72) Inventors: Tim BARRETT, Stevenage (GB); Richard Jonathan Daniel HATLEY, Stevenage (GB); Simon John Fawcett MACDONALD, Stevenage (GB); Paula SAKLATVALA, Stevenage (GB); Sing Yuen Eric TSE, Stevenage (GB)

(21) Appl. No.: 16/086,265

(22) PCT Filed: Mar. 16, 2017
(86) PCT No.: PCT/EP2017/056204

§ 371 Date: Sep. 18, 2018

(30) Foreign Application Priority Data
Mar. 18, 2016 (GB) 1604589.0

(51) Int. Cl. C07D 471/04

(52) U.S. Cl. CPC C07D 471/04; A61K 9/0053

(57) ABSTRACT

The invention relates to a compound which is (S)-4-((S)-3-Fluoro-3-(2-(5,6,7,8-tetrahydro-1, 8-naphthyridin-2-yl)ethyl)pyrrolidin-1-yl)-3-(3-(2-methoxyethoxy)phenyl) butanoic acid (1:1) citrate salt, pharmaceutical compositions including such compound, and to the use of such compound in therapy, including in the treatment of a disease or condition for which an αᵥJ36 integrin antagonist is indicated, and in particular idiopathic pulmonary fibrosis.

### FIELD OF THE INVENTION

The present invention relates to a pyrrolidine compound being an αᵥβ₆ integrin antagonist, pharmaceutical compositions comprising such compound and to its use in therapy, especially in the treatment of conditions for which an αᵥβ₆ integrin antagonist is indicated, to the use of a compound in the manufacture of a medicament for the treatment of conditions in which an antagonist of αᵥβ₆ integrin is indicated, and a method for the treatment of disorders in which antagonism of αᵥβ₆ integrin is indicated in a human.

### BACKGROUND OF THE INVENTION

Integrin superfamily proteins are heterodimeric cell surface receptors, composed of an alpha and beta subunit. At least 18 alpha and 8 beta subunits have been reported, which have been demonstrated to form 24 distinct alpha/beta heterodimers. Each chain comprises a large extracellular domain (>640 amino acids for the beta subunit, >940 amino acids for the alpha subunit), with a transmembrane spanning region of around 20 amino acids per chain, and generally a short cytoplasmic tail of 30-50 amino acids per chain. Different integrins have been shown to participate in a plethora of cellular biologies, including cell adhesion to the extracellular matrix, cell-cell interactions, and effects on cell migration, proliferation, differentiation and survival (Barczyk et al, Cell and Tissue Research, 2010, 339, 269).

Integrin receptors interact with binding proteins via short protein-protein binding interfaces. The integrin family can be grouped into sub-families that share similar binding recognition motifs in such ligands. A major subfamily is the RGD-integrins, which recognise ligands that contain an RGD (arginine-glycine-aspartic acid) motif within their protein sequence. There are 8 integrins in this sub-family, namely αᵥβ₁, αᵥβ₃, αᵥβ₅, αᵥ₆, αᵥβ₈, α_{IIb}β₃, α₅β₁, where nomenclature demonstrates that αᵥβ₁, αᵥβ₃, αᵥβ₅, αᵥβ₆, & αᵥβ₈ share a common αᵥ subunit with a divergent β subunit, and αᵥβ₁, α₅β₁ αᵥβ₁ & α₈β₁ share a common β₁ subunit with a divergent a subunit. The β₁ subunit has been shown to pair with 11 different a subunits, of which only the 3 listed above commonly recognise the RGD peptide motif (Humphries et al, Journal of Cell Science, 2006, 119, 3901).

The 8 RGD-binding integrins have different binding affinities and specificities for different RGD-containing ligands. Ligands include proteins such as fibronectin, vitronectin, osteopontin, and the latency associated peptides (LAPs) of Transforming Growth Factor β₁ and β₃ (TGFβ₁ and TGFβ₃). Integrin binding to the LAPs of TGFβ₁ and TGFβ₃ has been shown in several systems to enable activation of the TGFβ₁ and TGFβ₃ biological activities, and subsequent TGFβ-driven biologies (Worthington et al, Trends in Biochemical Sciences, 2011, 36, 47). The diversity of such ligands, coupled with expression patterns of RGD-binding integrins, generates multiple opportunities for disease intervention. Such diseases include fibrotic diseases (Margadant et al, EMBO reports, 2010, 11, 97), inflammatory disorders, cancer (Desgrosellier et al, Nature Reviews Cancer, 2010, 10, 9), restenosis, and other diseases with an angiogenic component (Weis et al, Cold Spring. Harb. Perspect. Med. 2011, 1, a 006478).

A significant number of αᵥ integrin antagonists (Goodman et al, Trends in Pharmacological Sciences, 2012, 33, 405) have been disclosed in the literature including inhibitory antibodies, peptides and small molecules. For antibodies these include the pan-av antagonists Intetumumab and Abituzumab (Gras, Drugs of the Future, 2015, 40, 97), the selective αᵥβ3 antagonist Etaracizumab, and the selective αᵥβ₆ antagonist STX-100. Cilengitide is a cyclic peptide antagonist that inhibits both αᵥβ₃ and αᵥβ₅ and SB-267268 is an example of a compound (Wilkinson-Berka et al, Invest. Ophthalmol. Vis. Sci., 2006, 47, 1600), that inhibits both αᵥβ₃ and αᵥβ₅. Invention of compounds to act as antagonists of differing combinations of αᵥ integrins enables novel agents to be generated tailored for specific disease indications.

Pulmonary fibrosis represents the end stage of several interstitial lung diseases, including the idiopathic interstitial pneumonias, and is characterised by the excessive deposition of extracellular matrix within the pulmonary interstitium. Among the idiopathic interstitial pneumonias, idiopathic pulmonary fibrosis (IPF) represents the commonest and most fatal condition with a typical survival of 3 to 5 years following diagnosis. Fibrosis in IPF is generally progressive, refractory to current pharmacological intervention and inexorably leads to respiratory failure due to obliteration of functional alveolar units. IPF affects approximately 500,000 people in the USA and Europe.

There are in vitro experimental, animal and IPF patient immunohistochemistry data to support a key role for the epithelially restricted integrin, αᵥβ₆, in the activation of TGFβ1. Expression of this integrin is low in normal epithelial tissues and is significantly up-regulated in injured and inflamed epithelia including the activated epithelium in IPF. Targeting this integrin, therefore, reduces the theoretical possibility of interfering with wider TGFβ homeostatic roles. Partial inhibition of the αᵥβ₆ integrin by antibody blockade has been shown to prevent pulmonary fibrosis without exacerbating inflammation (Horan G S et al Partial inhibition of integrin αᵥβ₆ prevents pulmonary fibrosis without exacerbating inflammation. Am J Respir Crit Care Med 2008 177: 56-65). Outside of pulmonary fibrosis, αᵥβ₆ is also considered an important promoter of fibrotic disease of other organs, including liver and kidney (Reviewed in Henderson N C et al Integrin-mediated regulation of TGFβ in Fibrosis, Biochimica et Biophysica Acta—Molecular Basis of Disease 2013 1832:891-896), suggesting that an αᵥβ₆ antagonist could be effective in treating fibrotic diseases in multiple organs.

Consistent with the observation that several RGD-binding integrins can bind to, and activate, TGFβ, different αᵥ integrins have recently been implicated in fibrotic disease (Henderson N C et al Targeting of αᵥ integrin identifies a core molecular pathway that regulates fibrosis in several organs Nature Medicine 2013 Vol 19, Number 12: 1617-1627; Sarrazy V et al Integrins αvβ5 and αvβ3 promote latent TGF-β1 activation by human cardiac fibroblast contraction Cardiovasc Res 2014 102:407-417; Minagawa S et al Selective targeting of TGF-β activation to treat flbroinflammatory airway disease Sci Transl Med 2014 Vol 6, Issue 241: 1-14; Reed N I et al . The αᵥβ₁ integrin plays a critical in vivo role in tissue fibrosis Sci Transl Med 2015 Vol 7, Issue 288: 1-8). Therefore inhibitors against specific members of the RGD binding integrin families, or with specific selectivity fingerprints within the RGD binding integrin family, may be effective in treating fibrotic diseases in multiple organs.

SAR relationships of a series of integrin antagonists against αᵥβ₃ αᵥβ₅, αᵥβ₆ and αᵥβ₈ have been described (Macdonald, S J F et al. Structure activity relationships of αᵥ integrin antagonists for pulmonary fibrosis by variation in aryl substituents. ACS Med Chem Lett 2014, 5, 1207-1212. 19 September 2014).

WO 2016/046225 A1 (published 31 Mar. 2016) disclosed compounds of Formula

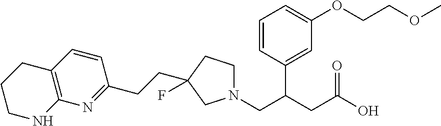 [SMILES: COCCOc1cccc(C(CC(=O)O)CN2CCC(F)(CCc3ccc4c(n3)NCCC4)C2)c1]

and salts thereof as αᵥβ₆ antagonists, including the specific diastereoisomer (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl) butanoic acid and a maleate and a citroconate salt thereof.

It is an object of the invention to provide αᵥβ₆ antagonists, including those with activities against other αᵥ integrins, such as αᵥβ₁, αᵥβ₃, αᵥβ₅ or αᵥβ₈, in particular an alternative salt of (S)-4-((S)-3-Fluoro-3-(2-(5,6,7,8-tetrahydro-1,8-naphthyridin-2-yl)ethyl)pyrrolidin-1-yl)-3-(3-(2-methoxyethoxy)phenyl)butanoic acid.

### BRIEF SUMMARY OF THE INVENTION

In a first aspect of the present invention, there is provided (S)-4-((S)-3-Fluoro-3-(2-(5,6,7,8-tetrahydro-1,8-naphthyridin-2-yl)ethyl)pyrrolidin-1-yl)-3-(3-(2-methoxyethoxy)phenyl)butanoic acid 1:1 citrate salt.

(S)-4-((S)-3-Fluoro-3-(2-(5,6,7,8-tetrahydro-1,8-naphthyridin-2-yl) ethyl)pyrrolidin-1-yl)-3-(3-(2-methoxyethoxy)phenyl)butanoic acid 1:1 citrate salt has αᵥβ₆ integrin antagonist activity and is believed to be of potential use for the treatment of certain disorders. The term αᵥβ₆ antagonist activity includes αᵥβ₆ inhibitor activity herein.

In a second aspect of the present invention, there is provided a pharmaceutical composition comprising (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid 1:1 citrate salt and a pharmaceutically acceptable carrier, diluent or excipient.

In a third aspect of the present invention, there is provided (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid 1:1 citrate salt for use in therapy, in particular in the treatment of a disease or condition for which an αᵥβ₆ integrin receptor antagonist is indicated.

In a fourth aspect of the present invention, there is provided a method of treatment of a disease or condition for which an αᵥβ₆ integrin receptor antagonist is indicated in a human in need thereof which comprises administering to a human in need thereof a therapeutically effective amount of (S)-4-((S)-3-Fluoro-3-(2-(5,6,7,8-tetrahydro-1,8-naphthyridin-2-yl)ethyl)pyrrolidin-1-yl)-3-(3-(2-methoxyethoxy) phenyl) butanoic acid 1:1 citrate salt.

In a fifth aspect of the present invention, there is provided the use of (S)-4-((S)-3-Fluoro-3-(2-(5,6,7,8-tetrahydro-1,8-naphthyridin-2-yl) ethyl) pyrrolidin-1-yl)-3-(3-(2-methoxyethoxy)phenyl)butanoic acid 1:1 citrate salt in the manufacture of a medicament for the treatment of a disease or condition for which an αᵥβ₆ integrin receptor antagonist is indicated.

### DETAILED DESCRIPTION OF THE INVENTION

In a first aspect of the present invention, there is provided (S)-4-((S)-3-Fluoro-3-(2-(5,6,7,8-tetrahydro-1,8-naphthyridin-2-yl)ethyl)pyrrolidin-1-yl)-3-(3-(2-methoxyethoxy)phenyl)butanoic acid 1:1 citrate salt (hereinafter also referred to as “the compound of the invention”).

It will be appreciated that many organic compounds can form complexes with solvents in which they are reacted or from which they are precipitated or crystallized. These complexes are known as “solvates”. For example, a complex with water is known as a “hydrate”. Solvents with high boiling points and/or capable of forming hydrogen bonds such as water, xylene, N-methyl pyrrolidinone, methanol and ethanol may be used to form solvates. Methods for identification of solvates include, but are not limited to, NMR and microanalysis. The compound of the invention may exist in solvated and unsolvated form.

The compound of the invention may be in crystalline or amorphous form. Furthermore, some of the crystalline forms of the compound of the invention may exist in different polymorphic forms. Polymorphic forms of the compound of the invention may be characterized and differentiated using a number of conventional analytical techniques, including, but not limited to, X-ray powder diffraction (XRPD) patterns, infrared (IR) spectra, Raman spectra, differential scanning calorimetry (DSC), thermogravimetric analysis (TGA) and solid state nuclear magnetic resonance (SSNMR).

The compound of the invention may also be prepared as an amorphous molecular dispersion in a polymer matrix, such as hydroxypropylmethyl cellulose acetate succinate, using a spray-dried dispersion (SDD) process to improve the stability and solubility of the drug substance.

The compound of the invention may also be delivered using a liquid encapsulation technology to improve properties such as bioavailability and stability, in either liquid or semi-solid filled hard capsule or soft gelatin capsule formats.

The compound of the invention may exist in one of several tautomeric forms. It will be understood that the present invention encompasses all tautomers of (S)-4-((S)-3-Fluoro-3-(2-(5,6,7,8-tetrahydro-1, 8-naphthyridin-2-yl) ethyl) pyrrolidin-1-yl)-3-(3-(2-methoxyethoxy) phenyl)butanoic acid in the form of a 1:1 citrate salt whether as individual tautomers or as mixtures thereof.

### Definitions

Terms are used within their accepted meanings. The following definitions are meant to clarify, but not limit, the terms defined.

As used herein, the term “treatment” refers to alleviating the specified condition, eliminating or reducing one or more symptoms of the condition, slowing or eliminating the progression of the condition, and delaying the reoccurrence of the condition in a previously afflicted or diagnosed patient or subject.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function.

### Compound Preparation

The compound of the invention may be made by a variety of methods, including standard chemistry.

It will be appreciated by those skilled in the art that the (E) or (Z) description of some intermediate compounds which can exist in two geometrical isomers, may contain the other geometric isomer as a minor component.

The compound of the invention may be prepared by reaction of a compound of Formula (I)

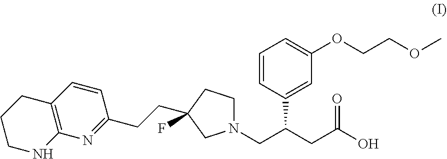 [SMILES: COCCOc1cccc([C@H](CC(=O)O)CN2CC[C@@](F)(CCc3ccc4c(n3)NCCC4)C2)c1]

with citric acid by methods well known to those skilled in the art.

A compound of Formula (I) may be prepared as disclosed in WO 2016/046225 A1 by a process involving deprotection of a compound of structural Formula (II), i.e. cleavage of the ester group:

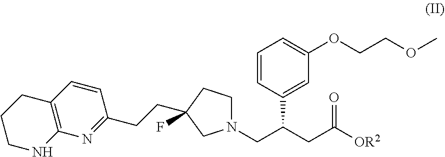

where R² is a C₁-C₆ alkyl group for example a tert-butyl, isopropyl, ethyl or methyl group. Alternatively R² is a chiral alkyl for example (−)-menthyl [from (1R, 2S, 5R)-2-isopropyl-5-methylcyclohexanol].

The deprotection of compound of structural Formula (II) where R² is methyl, menthyl or tert-butyl may be accomplished by acid hydrolysis using for example hydrochloric, hydrobromic, sulfuric, or trifluoroacetic acid, in an inert solvent, such as dichloromethane, 2-methyl-tetrahydrofuran, tetrahydrofuran, 1,4-dioxane, cyclopentyl methyl ether or water. Alternatively enzymatic hydrolysis may be used.

Alternatively the deprotection of compound of structural Formula (II) where R² is methyl, ethyl, isopropyl or menthyl may be accomplished by base hydrolysis using for example lithium hydroxide, sodium hydroxide, potassium hydroxide in a suitable solvent, e.g. an aqueous solvent such as aqueous methanol.

Compounds of Formula (II) may be obtained from compounds of Formula (III):

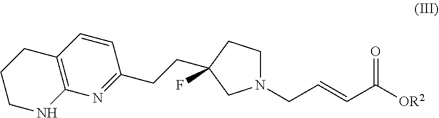

where R² is as defined above, by reaction with a boronic acid compound of structural Formula (IV):

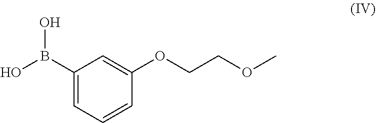

Alternatively a boronate ester, such as pinacol ester may be used, which provides the parent boronic acid in situ. Compounds of structural Formula (IV) are commercially αᵥ ailable e.g. from Enamine LLC, Princeton Corporate Plaza, 7 Deer Park Drive Ste. 17-3, Monmouth Jct. NJ (USA) 08852, Manchester Organics or Fluorochem. The reaction between the compounds of structural Formula (III) and (IV) may be performed in the presence of a suitable catalyst, such as a rhodium catalyst, for example the dimer of rhodium (1,5-cyclooctadiene)chloride, [Rh(COD)Cl]₂ and an additive such as a phosphine ligand, for example bis(diphenylphosphino)-1,1′-binaphthyl (BINAP), preferably in the presence of a base, such as aqueous potassium hydroxide, at elevated temperature, such as 50-90° C., and in a water-miscible solvent, such as 1,4-dioxane. The reaction is preferably carried out under strictly anaerobic conditions, where the reaction mixture is purged with an inert gas such as nitrogen, and evacuated under reduced pressure, repeating this process of evacuation and purging with nitrogen three times. The coupling reaction in the presence of (R)-BINAP provided a diastereoisomeric mixture with a predominant isomer, for example approximately 80:20 or higher. The predominant diastereoisomer when using (R)-BINAP has the (S) configuration (as similarly shown in respect of the preparation of structurally related compounds in WO02014/154725). The diastereoisomeric ratio may be further increased to, for example greater than 99:1, by chiral HPLC, chiral SFC, or by crystallisation, at either the ester stage (compound of Formula (II)) or after conversion to the corresponding acid (compound of Formula (I)). Use of enzymatic hydrolysis for the conversion of the compound of Formula (II) to the compound of Formula (I) may also be used to increase the diastereomeric ratio and may avoid the need to use methods such as chiral HPLC.

Compounds of Formula (III) may be obtained from compounds of Formula (V):

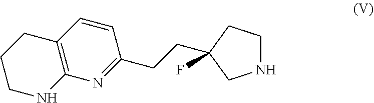 [SMILES: F[C@@]1(CCc2ccc3c(n2)NCCC3)CCNC1]

by reaction with a compound of Formula (VI)

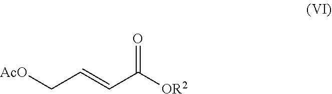

where R² is as defined above, in the presence of an organic base such as N,N-diisopropylethylamine (“DIPEA”) and a suitable palladium-based catalyst, for example PdCl₂(dppf)-CH₂Cl₂ [1,1′-bis(diphenylphosphino)ferrocene]dichloropalladium(II), complex with dichloromethane, in a solvent such as dichloromethane. The compound of Formula (VI) wherein R² represents tert-butyl is disclosed at page 32 of WO2014/154725. The compound of Formula (VI) wherein R² represents methyl is disclosed at page 50 of WO2014/15475. The compound of Formula (V) can be used as the parent compound, or be generated in situ from a salt, such as the dihydrochloride salt, in the presence of a tertiary amine base.

Compounds of Formula (VI) may be prepared by methods described herein. By way of illustration the compound of structural formula (VI), where R² is methyl, and the double bond having the (E) geometry, can be prepared by the method shown below, starting from the commercially available methyl 4-bromocrotonate and sodium or potassium acetate in acetonitrile at elevated temperature e.g. 50° C.:

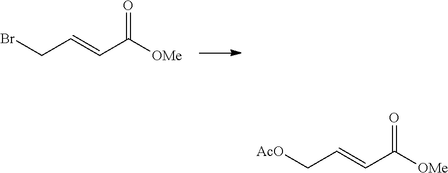 [SMILES: COC(=O)/C=C/CBr ; COC(=O)/C=C/COC(C)=O]

Compounds of Formula (V) may be prepared from compounds of structural Formula (VII):

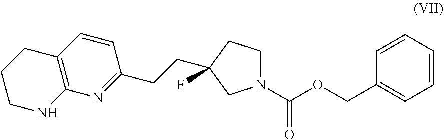 [SMILES: O=C(OCc1ccccc1)N1CC[C@@](F)(CCc2ccc3c(n2)NCCC3)C1]

by catalytic hydrogenolysis for example using a palladium catalyst deposited on carbon, in an inert solvent, such as ethanol or ethyl acetate.

Compounds of Formula (VII) may be obtained from compounds of Formula (VIII):

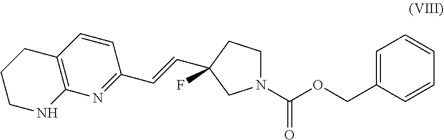 [SMILES: O=C(OCc1ccccc1)N1CC[C@@](F)(/C=C/c2ccc3c(n2)NCCC3)C1]

by diimide reduction, generated for example from benzenesulfonyl hydrazide in the presence of a base, such as potassium carbonate, in a suitable solvent, such as DMF, and at elevated temperature, such as 130° C.

Compounds of Formula (VIII) exist as geometrical isomers e.g. (E) or (Z)-form and may be used either as pure isomers or as mixtures. Compounds of Formula (VIII) may be obtained starting from known commercially αᵥ ailable (e.g. from Wuxi App Tec, 288 Fute Zhong Road, Waigaoquiao Free Trade, Shanghai 200131, China) compounds of Formula (IX):

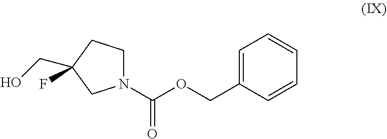 [SMILES: O=C(OCc1ccccc1)N1CC[C@@](F)(CO)C1]

which may be oxidised e.g. with sulphur trioxide in pyridine to the corresponding aldehyde of Formula (X):

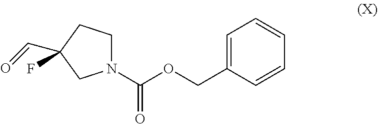 [SMILES: O=C[C@]1(F)CCN(C(=O)OCc2ccccc2)C1]

This compound of Formula (X) may then be reacted, which may be without isolation of the compound of Formula (X), with an ylide of Formula (XI):

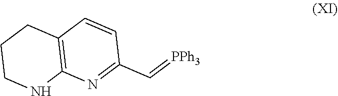

to thereby form the compound of Formula (VIII) which exists as a mixture of geometrical isomers (E) and (Z). It will be appreciated by those skilled in the art that there are other methods for forming the compound of Formula (VIII) from the aldehyde (X). The geometrical isomers can be separated by chromatography or used in the next step as a mixture. This overall scheme for preparation of the compound of Formula (I) is summarised below as Scheme (I):

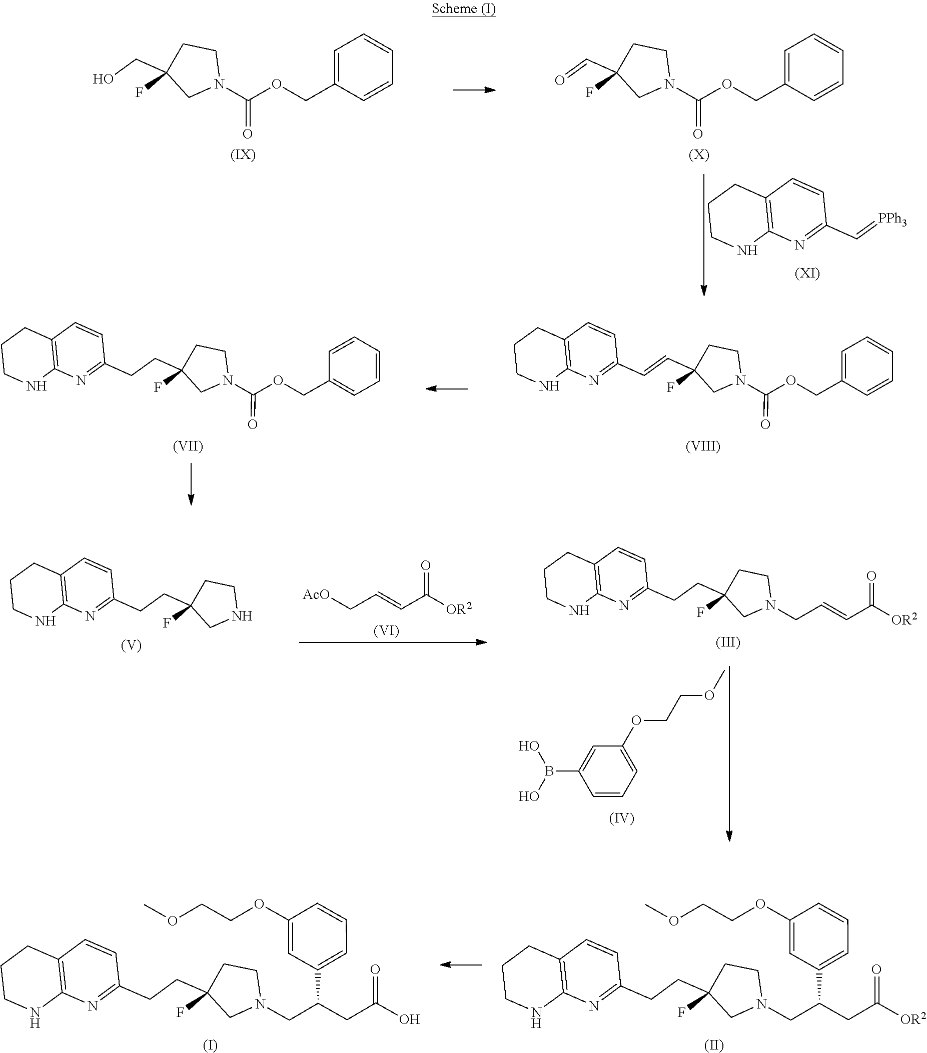

Ylide of Formula (XI) may be made starting from the compound of Formula (XII) (available from Fluorochem):

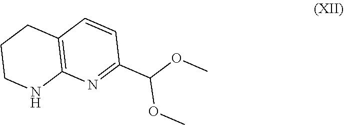

which by reaction with first hydrochloric acid followed by neutralisation with sodium bicarbonate may then be converted into an aldehyde of Formula (XIII):

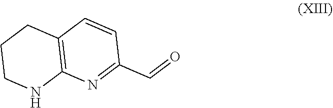 [SMILES: O=Cc1ccc2c(n1)NCCC2]

which may be reduced e.g. using sodium borohydride to the corresponding alcohol of Formula (XIV):

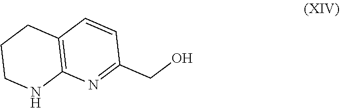 [SMILES: OCc1ccc2c(n1)NCCC2]

(see also the routes disclosed in US-A-20040092538 for preparation of alcohols of Formula (XIV)) which may then be brominated e.g. using phosphorus tribromide to produce the corresponding bromo compound of Formula (XV):

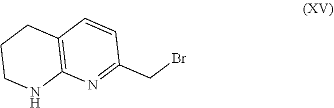 [SMILES: BrCc1ccc2c(n1)NCCC2]

which may be converted to the triphenylphosphonium bromide (XVI) by reacting with triphenylphosphine in a solvent such as acetonitrile.

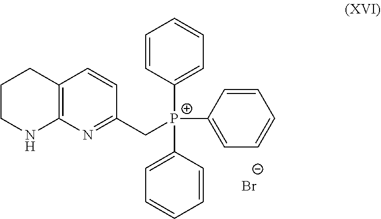 [SMILES: c1ccc([P+](Cc2ccc3c(n2)NCCC3)(c2ccccc2)c2ccccc2)cc1 ; [Br-]]

The above-mentioned ylide compound of Formula (XI) may be obtained by reaction of compound of structural Formula (XVI) with a base, such as a solution of potassium tert-butoxide in an inert solvent, such as THF. The ylide of Formula (XI) may be isolated or preferably formed in situ and reacted in the same vessel with an aldehyde of Formula (X) without prior isolation.

This overall scheme for preparation of ylide of Formula (XI) is summarised below as Scheme (II):

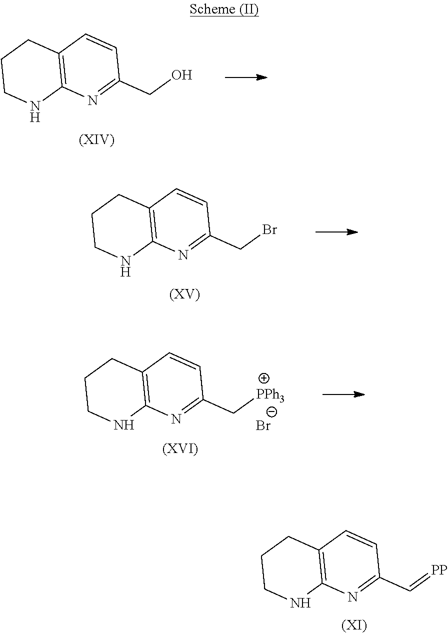

It will be appreciated that in any of the routes described above it may be advantageous to protect one or more functional groups. Examples of protecting groups and the means for their removal can be found in T. W. Greene ‘Protective Groups in Organic Synthesis’ (3rd edition, J. Wiley and Sons, 1999). Suitable amine protecting groups include acyl (e.g. acetyl), carbamate (e.g. 2′,2′,2′-trichloroethoxycarbonyl, benzyloxycarbonyl or t-butoxycarbonyl) and arylalkyl (e.g. benzyl), which may be removed by hydrolysis (e.g. using an acid such as hydrochloric acid in dioxane or trifluoroacetic acid in dichloromethane) or reductively (e.g. hydrogenolysis of a benzyl or benzyloxycarbonyl group or reductive removal of a 2′,2′,2′-trichloroethoxycarbonyl group using zinc in acetic acid) as appropriate. Other suitable amine protecting groups include trifluoroacetyl (—COCF₃) which may be removed by base catalysed hydrolysis.

It will be appreciated that in any of the routes described above, the precise order of the synthetic steps by which the various groups and moieties are introduced into the molecule may be varied. It will be within the skill of the practitioner in the art to ensure that groups or moieties introduced at one stage of the process will not be affected by subsequent transformations and reactions, and to select the order of synthetic steps accordingly.

The absolute configuration of the compound of Formula (I) may be obtained following an independent enantioselective synthesis from an intermediate of known absolute configuration. Alternatively an enantiomerically pure compound of Formula (I) may be converted into a compound whose absolute configuration is known. In either case comparison of spectroscopic data, optical rotation and retention times on an analytical HPLC column may be used to confirm absolute configuration. A third option where feasible is determination of absolute configuration through X-Ray crystallography.

### Methods of Use

The compound of the invention has αᵥ integrin antagonist activity, particularly αᵥβ₆ receptor activity, and thus has potential utility in the treatment of diseases or conditions for which an αᵥβ₆ antagonist is indicated.

The present invention thus provides (S)-4-((S)-3-Fluoro-3-(2-(5,6,7,8-tetrahydro-1,8-naphthyridin-2-yl) ethyl) pyrrolidin-1-yl)-3-(3-(2-methoxyethoxy)phenyl)butanoic acid 1:1 citrate salt for use in therapy. The compound of the invention can be for use in the treatment of a disease or condition for which an αᵥβ₆ integrin antagonist is indicated.

The present invention thus provides (S)-4-((S)-3-Fluoro-3-(2-(5,6,7,8-tetrahydro-1,8-naphthyridin-2-yl) ethyl) pyrrolidin-1-yl)-3-(3-(2-methoxyethoxy)phenyl)butanoic acid 1:1 citrate salt for use in the treatment of a disease or condition for which an αᵥβ₆ integrin antagonist is indicated.

Also provided is the use of (S)-4-((S)-3-Fluoro-3-(2-(5,6,7,8-tetrahydro-1,8-naphthyridin-2-yl) ethyl) pyrrolidin-1-yl)-3-(3-(2-methoxyethoxy)phenyl)butanoic acid 1:1 citrate salt in the manufacture of a medicament for the treatment of a disease or condition for which an αᵥβ₆ integrin antagonist is indicated.

Also provided is a method of treating a disease or condition for which an αᵥβ₆ integrin antagonist is indicated in a subject in need thereof which comprises administering a therapeutically effective amount of (S)-4-((S)-3-Fluoro-3-(2-(5,6,7,8-tetrahydro-1,8-naphthyridin-2-yl)ethyl)pyrrolidin-1-yl)-3-(3-(2-methoxyethoxy) phenyl) butanoic acid 1:1 citrate salt.

Suitably the subject in need thereof is a mammal, particularly a human.

Fibrotic diseases involve the formation of excess fibrous connective tissue in an organ or tissue in a reparative or reactive process. αᵥβ₆ antagonists are believed to be useful in the treatment of a variety of such diseases or conditions including those dependent on αᵥβ₆ integrin function and on activation of transforming growth factor beta via alpha v integrins. Accordingly, in one embodiment the disease or condition for which an αᵥβ₆ antagonist is indicated is a fibrotic disease. Diseases may include but are not limited to pulmonary fibrosis (e.g. idiopathic pulmonary fibrosis, non-specific interstitial pneumonia (NSIP), usual interstitial pneumonia (UIP), Hermansky-Pudlak syndrome, progressive massive fibrosis (a complication of coal workers' pneumoconiosis), connective tissue disease-related pulmonary fibrosis, airway fibrosis in asthma and COPD, ARDS associated fibrosis, acute lung injury, radiation-induced fibrosis, familial pulmonary fibrosis, pulmonary hypertension); renal fibrosis (diabetic nephropathy, IgA nephropathy, lupus nephritis, focal segmental glomerulosclerosis (FSGS), transplant nephropathy, autoimmune nephropathy, drug-induced nephropathy, hypertension-related nephropathy, nephrogenic systemic fibrosis); liver fibrosis (virally-induced fibrosis (e.g. hepatitis C or B), autoimmune hepatitis, primary biliary cirrhosis, alcoholic liver disease, non-alcoholic fatty liver disease including non-alcoholic steatohepatitis (NASH), congential hepatic fibrosis, primary sclerosing cholangitis, drug-induced hepatitis, hepatic cirrhosis); skin fibrosis (hypertrophic scars, scleroderma, keloids, dermatomyositis, eosinophilic fasciitis, Dupytrens contracture, Ehlers-Danlos syndrome, Peyronie's disease, epidermolysis bullosa dystrophica, oral submucous fibrosis); ocular fibrosis (age-related macular degeneration (AMD), diabetic macular oedema, dry eye, glaucoma) corneal scarring, corneal injury and corneal wound healing, prevention of filter bleb scarring post trabeculectomy surgery; cardiac fibrosis (congestive heart failure, atherosclerosis, myocardial infarction, endomyocardial fibrosis, hypertrophic cardiomyopathy (HCM)) and other miscellaneous fibrotic conditions (mediastinal fibrosis, myelofibrosis, retroperitoneal fibrosis, Crohn's disease, neurofibromatosis, uterine leiomyomas (fibroids), chronic organ transplant rejection. There may be further benefit from additional inhibition of αᵥβ₁, αᵥβ₅ or αᵥβ₈ integrins.

In addition, pre-cancerous lesions or cancers associated with αᵥβ₆ integrins may also be treated (these may include but are not limited to endometrial, basal cell, liver, colon, cervical, oral, pancreas, breast and ovarian cancers, Kaposi's sarcoma, Giant cell tumours and cancer associated stroma). Conditions that may derive benefit from effects on angiogenesis may also benefit (e.g. solid tumours).

The term “disease or condition for which an αᵥβ₆ antagonist is indicated”, is intended to include any or all of the above disease states.

In one embodiment the disease or condition for which an αᵥβ₆ antagonist is indicated is idiopathic pulmonary fibrosis.

In another embodiment the disease or condition for which an αᵥβ₆ antagonist is indicated is selected from corneal scarring, corneal injury and corneal wound healing.

### Compositions

While it is possible that for use in therapy, (S)-4-((S)-3-Fluoro-3-(2-(5,6,7,8-tetrahydro-1,8-naphthyridin-2-yl) ethyl) pyrrolidin-1-yl)-3-(3-(2-methoxyethoxy) phenyl) butanoic acid 1:1 citrate salt may be administered as the raw chemical, it is common to present the active ingredient as a pharmaceutical composition.

The present invention therefore provides in a further aspect a pharmaceutical composition comprising (S)-4-((S)-3-Fluoro-3-(2-(5,6,7,8-tetrahydro-1,8-naphthyridin-2-yl) ethyl) pyrrolidin-1-yl)-3-(3-(2-methoxyethoxy) phenyl) butanoic acid 1:1 citrate salt and a pharmaceutically acceptable carrier, diluent or excipient. The carrier, diluent or excipient must be acceptable in the sense of being compatible with the other ingredients of the composition and not deleterious to the recipient thereof.

In accordance with another aspect of the invention there is also provided a process for the preparation of a pharmaceutical composition including admixing the compound of the invention with a pharmaceutically acceptable carrier, diluent or excipient. The pharmaceutical composition can be for use in the treatment of any of the conditions described herein.

Further provided is a pharmaceutical composition for the treatment of diseases or conditions for which an αᵥβ₆integrin antagonist is indicated comprising (S)-4-((S)-3-Fluoro-3-(2-(5,6,7,8-tetrahydro-1,8-naphthyridin-2-yl) ethyl) pyrrolidin-1-yl)-3-(3-(2-methoxyethoxy)phenyl) butanoic acid 1:1 citrate salt.

Further provided is a pharmaceutical composition comprising 0.05 to 1000mg of (S)-4-((S)-3-Fluoro-3-(2-(5,6,7,8-tetrahydro-1,8-naphthyridin-2-yl) ethyl) pyrrolidin-1-yl)-3-(3-(2-methoxyethoxy)phenyl)butanoic acid 1:1 citrate salt and 0.1 to 2g of a pharmaceutically acceptable carrier, diluent or excipient.

Since the compound of the invention is intended for use in pharmaceutical compositions it will be readily understood that it is preferably provided in substantially pure form, for example, at least 60% pure, more suitably at least 75% pure and preferably at least 85% pure, especially at least 98% pure (% in a weight for weight basis).

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose (for administration more than once a day), as herein above recited, or an appropriate fraction thereof, of an active ingredient.

Pharmaceutical compositions may be adapted for administration by any appropriate route, for example by the oral (including buccal or sublingual), rectal, inhaled, intranasal, topical (including buccal, sublingual or transdermal), vagina, ocular or parenteral (including subcutaneous, intramuscular, intravenous or intradermal) route. Such compositions may be prepared by any method known in the art of pharmacy, for example by bringing into association the active ingredient with the carrier or excipient.

In one embodiment the pharmaceutical composition is adapted for oral administration. Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders suitable for incorporating into tablets or capsules may be prepared by reducing the compound to a suitable fine particle size (e.g. by micronisation) and mixing with a similarly prepared pharmaceutical carrier such as an edible carbohydrate, as, for example, starch or mannitol. Flavoring, preservative, dispersing and coloring agent can also be present.

Capsules may be made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation.

A disintegrating or solubilising agent such as agaragar, calcium carbonate or sodium carbonate can also be added to improve the availability of the medicament when the capsule is ingested.

Moreover, when desired or necessary, suitable binders, glidants, lubricants, sweetening agents, flavours, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like.

Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavour additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release as for example by coating or embedding particulate material in polymers, wax or the like. The compound of the invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compound of the invention may also be prepared as an amorphous molecular dispersion in a polymer matrix, such as hydroxypropylmethyl cellulose acetate succinate, using a spray-dried dispersion (SDD) process to improve the stability and solubility of the drug substance.

The compound of the invention may also be delivered using a liquid encapsulation technology to improve properties such as bioavailability and stability, in either liquid or semi-solid filled hard capsule or soft gelatin capsule formats. Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils. For treatments of the eye or other external tissues, for example mouth and skin, the compositions are preferably applied as a topical ointment or cream. When formulated in an ointment, the active ingredient may be employed with either a paraffinic or a water miscible ointment base. Alternatively, the active ingredient may be formulated in a cream with an oil-in-water cream base or a water-in-oil base. The compounds of this invention can be administered as topical eye drops. The compound of this invention can be administered via sub-conjunctival, intracameral or intravitreal routes which would necessitate administration intervals that are longer than daily.

Pharmaceutical formulations adapted for topical administrations to the eye include eye drops wherein the active ingredient is dissolved or suspended in a suitable carrier, especially an aqueous solvent. Formulations to be administered to the eye will have ophthalmically compatible pH and osmolality. One or more ophthalmically acceptable pH adjusting agents and/or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, and sodium lactate; and buffers such as citrate/dextrose, sodium bicarbonate and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulflte anions.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodable polymers (e.g. poly(ethylene vinyl) acetate (EVA), superhydrolyzed PVA), hydroxyalkyl cellulose (HPC), methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition and polymeric coatings that will enhance drug diffusion, erosion, dissolution and osmosis.

Formulations for drug delivery using ocular devices may combine one or more active agents and adjuvants appropriate for the indicated route of administration. For example, the active agents may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers and a carrier or diluent that has a time delay property. Representative examples of biodegradable compositions can include albumin, gelatin, starch, cellulose, dextrans, polysaccharides, poly (D, L-lactide), poly (D, L-lactide-co-glycolide), poly (glycolide), poly (hydroxybutyrate), poly (alkylcarbonate) and poly (orthoesters) and mixtures thereof. Representative examples of non-biodegradable polymers can include EVA copolymers, silicone rubber and poly (methylacrylate), and mixtures thereof.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig (2005) Adv. Drug Deliv. Rev. 3; 57:1595-639, herein incorporated by reference for purposes of its teachings of examples of polymers for use in ocular drug delivery.

Pharmaceutical compositions adapted for topical administration in the mouth include lozenges, pastilles and mouth washes.

Pharmaceutical compositions adapted for rectal administration may be presented as suppositories or as enemas.

Dosage forms for nasal or inhaled administration may conveniently be formulated as aerosols, solutions, suspensions, gels or dry powders.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solutions which may contain anti-oxidants, buffers, bacteriostats and solutes which render the composition isotonic with the blood of the intended recipient, and aqueous and non-aqueous sterile suspensions which may include suspending agents and thickening agents. The compositions may be presented in unitdose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The compound of the invention may be administered in a long-acting parenteral (LAP) drug delivery system. Such drug delivery systems include formulations which aim to provide a slow release of drug once injected. LAP formulations may be particulate based, e.g. nano or micron sized polymeric spherical particles, which once injected would not be retrieved thus acting as a depot formulation; or small rod-like insert devices which may be retrieved if needed. Long acting particulate injectable formulations may be composed of an aqueous suspension of crystalline drug particle, where the drug has low solubility, thus providing a slow dissolution rate. Polymeric based LAP formulations are typically composed of a polymer matrix containing a drug (of hydrophilic or hydrophobic nature) homogeneously dispersed within the matrix. When LAP formulations are polymer based, the polymer widely used is poly-d,l-lactic-co-glycolic acid (PLGA) or versions thereof.

A therapeutically effective amount of a compound of the invention will depend upon a number of factors including, for example, the age and weight of the subject, the precise condition requiring treatment and its severity, the nature of the formulation, and the route of administration, and will ultimately be at the discretion of the attendant physician or veterinarian.

In the pharmaceutical composition, each dosage unit for oral or parenteral administration may contain from 0.01 to 3000 mg, or 0.1 to 2000mg, or more typically 0.5 to 1000 mg of a compound of the invention calculated as the zwitterion parent compound.

Each dosage unit for nasal or inhaled administration preferably contains from 0.001 to 50 mg, more preferably 0.01 to 5 mg, yet more preferably 1 to 50 mg, of a compound of the invention, calculated as the zwitterion parent compound.

For administration of a nebulised solution or suspension, a dosage unit typically contains from 1 to 15mg which may suitably be delivered once daily, twice daily or more than twice daily. The compound of the invention may be provided in a dry or lyophilised powder for reconstitution in the pharmacy or by the patient, or may, for example, be provided in an aqueous saline solution.

The compound of the invention can be administered in a daily dose (for an adult patient) of, for example, an oral or parenteral dose of 0.01 mg to 3000 mg per day, or 0.5 to 1000 mg per day or 0.5 to 300mg per day, or 2 to 300 mg per day, or a nasal or inhaled dose of 0.001 to 50 mg per day or 0.01 to 50 mg per day, or 1 to 50mg per day, of the compound of the invention, calculated as the zwitterion parent compound. This amount may be given in a single dose per day or more usually in a number (such as two, three, four, five or six) of sub-doses per day such that the total daily dose is the same. An effective amount of (S)-4-((S)-3-Fluoro-3-(2-(5,6,7,8-tetrahydro-1,8-naphthyridin-2-yl)ethyl)pyrrolidin-1-yl)-3-(3-(2-methoxyethoxy) phenyl) butanoic acid 1:1 citrate salt may be determined as a proportion of the effective amount of the zwitterion parent compound.

The compound of the invention may be employed alone or in combination with other therapeutic agents. Combination therapies according to the present invention thus comprise the administration of the compound of the invention, and the use of at least one other pharmaceutically active agent. Preferably, combination therapies according to the present invention comprise the administration of the compound of the invention, and at least one other pharmaceutically active agent. The compound of the invention and the other pharmaceutically active agent(s) may be administered together in a single pharmaceutical composition or separately and, when administered separately this may occur simultaneously or sequentially in any order. The amounts of the compound of the invention and the other pharmaceutically active agent(s) and the relative timings of administration will be selected in order to achieve the desired combined therapeutic effect.

Thus in a further aspect, there is provided a combination comprising a compound of the invention and at least one other pharmaceutically active agent.

Thus in one aspect, the compound and pharmaceutical compositions according to the invention may be used in combination with or include one or more other therapeutic agents, including therapies for allergic disease, inflammatory disease, autoimmune disease, anti-fibrotic therapies and therapies for obstructive airway disease, therapies for diabetic ocular diseases, and therapies for corneal scarring, corneal injury and corneal wound healing.

Anti-allergic therapies include antigen immunotherapy (such as components and fragments of bee venom, pollen, milk, peanut, CpG motifs, collagen, other components of extracellular matrix which may be administered as oral or sublingual antigens), anti-histamines (such as cetirizine, loratidine, acrivastine, fexofenidine, chlorphenamine), and corticosteroids (such as fluticasone propionate, fluticasone furoate, beclomethasone dipropionate, budesonide, ciclesonide, mometasone furoate, triamcinolone, flunisolide, prednisolone, hydrocortisone).

Anti-inflammatory therapies include NSAIDs (such as aspirin, ibuprofen, naproxen), leukotriene modulators (such as montelukast, zafirlukast, pranlukast), and other anti-inflammatory therapies (such as iNOS inhibitors, tryptase inhibitors, IKK2 inhibitors, p38 inhibitors (losmapimod, dilmapimod), elastase inhibitors, beta2 agonists, DP1 antagonists, DP2 antagonists, pI3K delta inhibitors, ITK inhibitors, LP (lysophosphatidic) inhibitors or FLAP (5-lipoxygenase activating protein) inhibitors (such as sodium 3-(3-(tert-butylthio)-1-(4-(6-ethoxypyridin-3-yl)benzyl)-5-((5-methylpyridin-2-yl)methoxy)-1H-indol-2-yl)-2,2-dimethylpropanoate); adenosine ata agonists (such as adenosine and regadenoson), chemokine antagonists (such as CCR3 antagonists or CCR4 antagonists), mediator release inhibitors.

Therapies for autoimmune disease include DMARDS (such as methotrexate, leflunomide, azathioprine), biopharmaceutical therapies (such as anti-IgE, anti-TNF, anti-interleukins (such as anti-IL-1, anti-IL-6, anti-IL-12, anti-IL-17, anti-IL-18), receptor therapies (such as etanercept and similar agents); antigen non-specific immunotherapies (such as interferon or other cytokines/chemokines, cytokine/chemokine receptor modulators, cytokine agonists or antagonists, TLR agonists and similar agents).

Other anti-fibrotic therapies includes inhibitors of TGFp synthesis (such as pirfenidone), tyrosine kinase inhibitors targeting the vascular endothelial growth factor (VEGF), platelet-derived growth factor (PDGF) and fibroblast growth factor (FGF) receptor kinases (such as Nintedanib (BIBF-1120) and imatinib mesylate (Gleevec)), endothelin receptor antagonists (such as ambrisentan or macitentan), antioxidants (such as N-acetylcysteine (NAC); broad-spectrum antibiotics (such as cotrimoxazole, tetracyclines (minocycline hydrochloride)), phosphodiesterase 5 (PDES) inhibitors (such as sildenafil), anti-avl3x antibodies and drugs (such as anti-αᵥβ₆ monoclonal antibodies such as those described in WO2003100033A2 may be used in combination, intetumumab, cilengitide) may be used in combination.

Therapies for obstructive airway diseases include bronchodilators such as short-acting β2-agonists, such as salbutamol), long-acting β2-agonists (such as salmeterol, formoterol and vilanterol), short-acting muscarinic antagonists (such as ipratropium bromide), long-acting muscarinic antagonists, (such as tiotropium, umeclidinium).

In some embodiments, treatment can also involve combination of the compound of the invention with other existing modes of treatment, for example existing agents for treatment of diabetic ocular diseases, such as anti VEGF therapeutics e.g. Lucentis®, Avastin®, and Aflibercept• and steroids, e.g., triamcinolone, and steroid implants containing fluocinolone acetonide. In some embodiments, treatment can also involve combination of the compound of the invention with other existing modes of treatment, for example existing agents for treatment of corneal scarring, corneal injury or corneal wound healing, such as Gentel®, calf blood extract, Levofloxacin®, and Ofloxacin®.

The compound and compositions of the invention may be used to treat cancers alone or in combination with cancer therapies including chemotherapy, radiotherapy, targeted agents, immunotherapy and cell or gene therapy.

The combinations referred to above may conveniently be presented for use in the form of a pharmaceutical composition and thus pharmaceutical compositions comprising a combination as defined above together with a pharmaceutically acceptable diluent or carrier represent a further aspect of the invention. The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

It will be appreciated that when the compound of the present invention is administered in combination with one or more other therapeutically active agents normally administered by the inhaled, intravenous, oral, intranasal, ocular topical or other route that the resultant pharmaceutical composition may be administered by the same route. Alternatively, the individual components of the composition may be administered by different routes.

The present invention will now be illustrated by way of example only.

### Abbreviations

The following list provides definitions of certain abbreviations as used herein. It will be appreciated that the list is not exhaustive, but the meaning of those abbreviations not herein below defined will be readily apparent to those skilled in the art.
Ac (acetyl)
BCECF-AM (2′,7-bis-(2-carboxyethyl)-5-(and-6)-carboxyfluorescein acetoxymethyl ester)
BEH (Ethylene Bridged Hybrid Technology)
Bu (butyl)
CBZ (carboxybenzyl)
CHAPS (3-[(3-Cholamidopropyl)dimethylammonio]-1-propanesulfonate)
Chiralcel OD-H (cellulose tris(3,5-dimethylphenylcarbamate) coated on 5 μm silica gel)
Chiralpak AD-H (amylose tris(3,5-dimethylphenylcarbamate) coated on 5 μm silica gel)
Chiralpak ID (amylose tris(3-chlorophenylcarbamate) immobilised on 5 μm silica gel)
Chiralpak AS (amylose tris((S)-alpha-methylbenzylcarbamate) coated on 5 μm silica gel)
CDI (carbonyl diimidazole)
CSH (Charged Surface Hybrid Technology)
CV (column volume)
DCM (dichloromethane)
DIPEA (diisopropylethylamine)
DMF (N,N-dimethylformamide)
DMSO (dimethylsulfoxide)
DSC (differential scanning colorimetry)
Et (ethyl)
EtOH (ethanol)
EtOAc (ethyl acetate)
h (hour/hours)
HCl (Hydrochloric acid)
HEPES (4-(2-hydroxyethyl)-1-piperazineethanesulfonic acid)
LCMS (liquid chromatography mass spectrometry)
MDAP (mass directed auto-preparative HPLC)
MDCK (Madin-Darby canine kidney)
Me (methyl)
MeCN (acetonitrile)
MeOH (methanol)
MS (mass spectrum)
min minute/minutes
PdCl₂(dppf)-CH₂Cl₂ [1,1′-Bis(diphenylphosphino)ferrocene]dichloropalladium(II), complex with
dichloromethane
Ph (phenyl)
ⁱPr (isopropyl)
(R)-BINAP (R)-(+)-2,2′-bis(diphenylphosphino)-1,1′-binaphthalene
[Rh(COD)Cl]₂ ((chloro(1,5-cyclooctadiene)rhodium(I) dinner)
RT (Retention Time)
SPE (solid phase extraction)
TBME (tert-butyl methyl ether)
TEA (triethylamine)
TFA (trifluoroacetic acid)
TGA (thermal gravimetric analysis)
THF (tetrahydrofuran)
TLC (thin layer chromatography)
UPLC (Ultra Performance Liquid Chromatography)

All references to brine refer to a saturated aqueous solution of sodium chloride.

### Experimental Details

### Analytical LCMS

Analytical LCMS was conducted on one of the following systems A, B, C or D.

The UV detection to all systems was an averaged signal from wavelength of 220 nm to 350 nm and mass spectra were recorded on a mass spectrometer using alternate-scan positive and negative mode electrospray ionization.

Experimental details of LCMS systems A-D as referred to herein are as follows:

System A

Column: 50 mm×2.1 mm ID, 1.7 μm Acquity UPLC BEH C₁₈ column

Flow Rate: 1 mL/min.

Temp.: 40° C.

Solvents: A: 10 mM ammonium bicarbonate in water adjusted to pH10 with ammonia solution
B: Acetonitrile

| | | | |
|---|---|---|---|
| Gradient: | Time (min) | A % | B % |

| | | |
|---|---|---|
| 0 | 99 | 1 |
| 1.5 | 3 | 97 |
| 1.9 | 3 | 97 |
| 2.0 | 99 | 1 |

System B

Column: 50 mm×2.1 mm ID, 1.7 μm Acquity UPLC BEH C18 column

Flow Rate: 1 mL/min

Temp.: 40° C.

Solvents: A: 0.1% v/v solution of formic acid in water
B: 0.1% v/v solution of formic acid in acetonitrile

| | | | |
|---|---|---|---|
| Gradient: | Time (min) | A % | B % |

| | | |
|---|---|---|
| 0 | 97 | 3 |
| 1.5 | 0 | 100 |
| 1.9 | 0 | 100 |
| 2.0 | 97 | 3 |

System C

Column: 50 mm×2.1 mm ID, 1.7 μm Acquity UPLC CSH C18 column

Flow Rate: 1 mL/min.

Temp.: 40° C.

Solvents: A: 10 mM ammonium bicarbonate in water adjusted to pH10 with ammonia solution
B: Acetonitrile

| | | | |
|---|---|---|---|
| Gradient: | Time (min) | A % | B % |

| | | |
|---|---|---|
| 0 | 97 | 3 |
| 1.5 | 5 | 95 |
| 1.9 | 5 | 95 |
| 2.0 | 97 | 3 |

System D

Column: 50 mm×2.1 mm ID, 1.7 μm Acquity UPLC BEH C18 column

Flow Rate: 1 mL/min

Temp.: 40° C.

Solvents: A: 0.1% v/v solution of trifluoroacetic acid in water
B: 0.1% v/v solution of trifluoroacetic acid in acetonitrile

| | | | |
|---|---|---|---|
| Gradient: | Time (min) | A % | B % |

| | | |
|---|---|---|
| 0 | 95 | 5 |
| 1.5 | 5 | 95 |
| 1.9 | 5 | 95 |
| 2.0 | 95 | 5 |

### Intermediate 1: 7-(Bromomethyl)-1,2,3,4-tetrahydro-1,8-naphthyridine (Compound (XV))

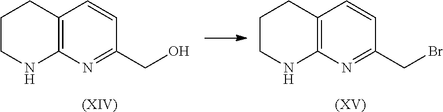

Phosphorus tribromide (0.565 mL, 5.99 mmol) was added dropwise to a suspension of (5,6,7,8-tetrahydro-1,8-naphthyridin-2-yl) methanol (Compound (XIV)): see US20040092538, page 80, [0844]) (820 mg, 4.99 mmol) in anhydrous acetonitrile (50 mL) at 0° C. under nitrogen. Upon addition a deep orange coloured precipitate formed, which turned to pale orange. The reaction mixture was stirred at 0° C. for 1 h by which time the reaction was complete. The mixture was concentrated in vacuo and the residue was partitioned between ethyl acetate (250 mL) and a saturated aqueous solution of NaHCO₃ (250 mL). The aqueous phase was further extracted with ethyl acetate (250 mL). The combined organic solutions were passed through a hydrophobic frit and then concentrated in vacuo to give the title compound (1.05 g, 93%)as a fluffy creamy solid: LCMS (System C) RT=0.95 min, ES+ve m/z 227, 229 (M+H)⁺.

### Intermediate 2: Triphenyl ((5,6,7,8-tetrahydro-1,8-naphthyridin-2-yl)methyl)phosphonium bromide (Compound (XVI))

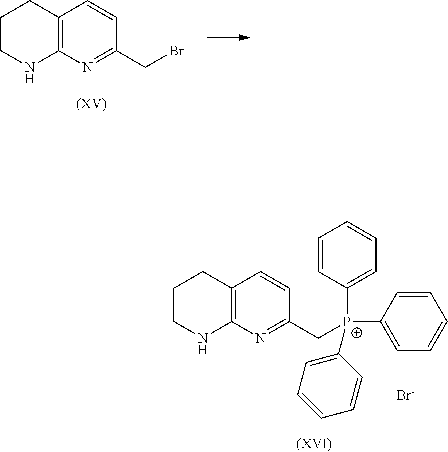

A solution of 7-(bromomethyl)-1,2,3,4-tetrahydro-1,8-naphthyridine (Compound (XV), Intermediate 1) (1.00 g, 4.40 mmol) in acetonitrile (98 mL) was treated with triphenylphosphine (1.270 g, 4.84 mmol) and the solution was stirred at room temperature under nitrogen overnight. The mixture was concentrated in vacuo to give a dark cream solid, which was then triturated with diethyl ether to give the title compound (2.139 g, 99%) as a pale cream solid: LCMS (System C) RT=1.23 min, ES+ve m/z 409 (M+H)⁺.

### Intermediate 3: (E, Z) Benzyl 3-fluoro-3-(2-(5,6,7,8-tetrahydro-1,8-naphthyridin-2-yl)vinyl)pyrrolidine-1-carboxylate. (Compound (VIII))

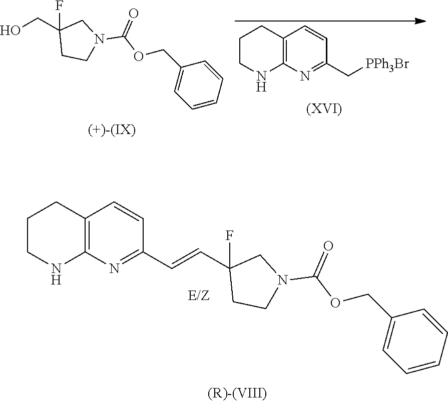

A stirred solution of (+)-benzyl 3-fluoro-3-(hydroxmethyl) pyrrolidine-1-carboxylate (Compound (IX): available from Wuxi App Tec; see also Tetrahedron Asymmetry, 27 (2016), pages 1222-1230) (260 mg, 1.03 mmol) in DCM (3 mL) and DMSO (0.3 mL), under nitrogen, was treated with DIPEA (0.896 mL, 5.13 mmol). After cooling to 0-5° C. (ice bath) pyridine sulfur trioxide (327 mg, 2.05 mmol) was added portionwise over ca. 5 min to oxidise the alcohol compound (IX) to the corresponding aldehyde compound (X) which was not isolated. The cooling bath was removed and stirring was continued for 0.5 h. Meanwhile a solution of triphenyl ((5,6,7,8-tetrahydro-1,8-naphthyridin-2-yl) methyl) phosphonium bromide (Compound (XVI), for a preparation see Intermediate 2) (553 mg, 1.13 mmol) in anhydrous DCM (10 mL), under nitrogen, was treated dropwise with potassium tert-butoxide (1M in THF) (1.232 mL, 1.232 mmol) over ca. 5 min resulting in an orange coloured solution. Stirring was continued for 10 min and then the aldehyde (Formula (X)) solution was added to the ylide solution in one shot and the mixture was stirred at ambient temperature for 22 h. The reaction mixture was diluted with DCM (20 mL), washed with saturated aqueous sodium bicarbonate (20 mL) and brine (20 mL), dried (Na₂SO₄) then evaporated in vacuo. The dark brown residue was purified by chromatography on a 20 g silica SPE cartridge and eluted with a gradient of 0-100% ethyl acetate-cyclohexane over 30 min to obtain the title compound as two geometrical isomers:

Isomer 1: a straw-coloured gum (123.4 mg, 31%), LCMS (System A) RT=1.28 min, 95%, ES+ve m/z 382 (M+H)⁺ and

Isomer 2: a straw-coloured gum (121.5 mg, 31%), LCMS (System A) RT=1.22 min, 91%, ES+ve m/z 382 (M+H)⁺

Overall yield=244.9 mg, 62.5%.

The configuration of Intermediate 3 was subsequently shown to be (R) and the two geometrical isomers are: (R,E)-benzyl 3-fluoro-3-(2-(5,6,7,8-tetrahydro-1,8-naphthyridin-2-yl)vinyl)pyrrolidine-1-carboxylate and (R,Z)-benzyl 3-fluoro-3-(2-(5,6,7,8-tetrahydro-1,8-naphthyridin-2-yl)vinyl)pyrrolidine-1-carboxylate.

### Intermediate 4: Benzyl 3-fluoro-3-(2-(5,6,7,8-tetra hydro-1,8-na phthyrid in-2-yl)ethyl)pyrrolidine-1-carboxylate (Compound (VII))

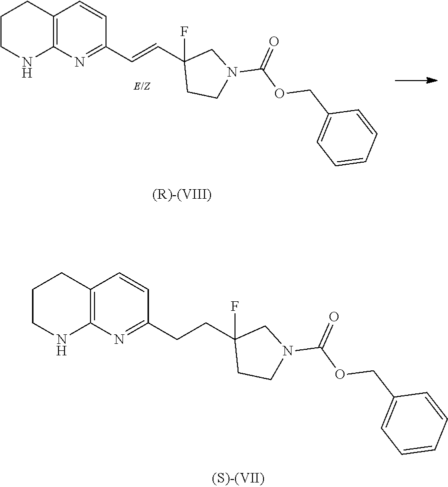 [SMILES: O=C(OCc1ccccc1)N1CCC(F)(/C=C/c2ccc3c(n2)NCCC3)C1 ; O=C(OCc1ccccc1)N1CCC(F)(CCc2ccc3c(n2)NCCC3)C1]

A solution of (E,Z)-benzyl 3-fluoro-3-(2-(5,6,7,8-tetrahydro-1,8-naphthyridin-2-yl)vinyl)pyrrolidine-1-carboxylate (Compound (VIII), Intermediate 3) (244 mg, 0.640 mmol) (1:1, E:Z) in DMF (2 mL) was treated with benzenesulfonyl hydrazide (available from Alfa Aesar) (275 mg, 1.60 mmol) and potassium carbonate (354 mg, 2.56 mmol). The reaction mixture was heated to 130 ° C. for 1 h, then allowed to cool and partitioned between DCM and water. The organic phase was washed with water and dried through a hydrophobic frit. The organic solution was evaporated in vacuo and the residual orange oil was purified by chromatography on a silica cartridge (20 g) eluting with a gradient of 0-50% [(3:1 EtOAc-EtOH)-EtOAc] over 20 min. The appropriate fractions were combined and evaporated in vacuo to give the title compound (150 mg, 61%) as a pale yellow gum: LCMS (System A) RT=1.24 min, 90%, ES+ve m/z 384 (M+H)⁺. The absolute configuration of Intermediate 4 was subsequently shown to be (S) hence the compound is (S)-benzyl 3-fluoro-3-(2-(5,6,7,8-tetrahydro-1,8-naphthyridin-2-yl)ethyl)pyrrolidine-1-carboxylate.

### Intermediate 5: 7-(2-(3-Fluoropyrrolidin-3-yl)ethyl)-1,2,3,4-tetrahydro-1,8-naphthyridine (Compound (V))

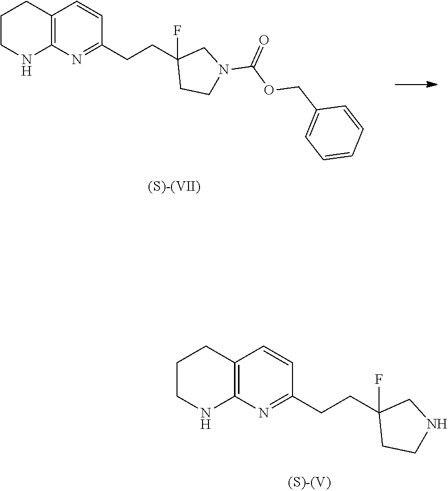 [SMILES: O=C(OCc1ccccc1)N1CCC(F)(CCc2ccc3c(n2)NCCC3)C1 ; FC1(CCc2ccc3c(n2)NCCC3)CCNC1]

A stirred solution of benzyl 3-fluoro-3-(2-(5,6,7,8-tetrahydro-1,8-naphthyridin-2-yl)ethyl)pyrrolidine-1-carboxylate (Compound (VII), Intermediate 4) (4.67 g, 12.2 mmol) in ethanol (70 mL) containing 10% palladium on carbon (0.50 g) was stirred under a hydrogen atmosphere for 7 h. LCMS showed incomplete deprotection and additional 10% palladium on carbon (0.25 g) was added and the mixture was stirred under a hydrogen atmosphere overnight. The reaction mixture existed as a dark grey suspension so DCM was added to dissolve up the material until the mixture became black. The catalyst was removed by filtration through a pad of celite and the filtrate and washings were evaporated in vacuo. The residue was evaporated from DCM to obtain the title compound as an orange oil (3.28 g): LCMS (System A) RT=0.79 min, 90%, ES+ve m/z 250 (M+H)⁺. The configuration of Intermediate 5 was subsequently established as (S) and the name of the compound is (S)-7-(2-(3-fluoropyrrolidin-3-ypethyl)-1,2,3,4-tetrahydro-1,8-naphthyridine.

### Intermediate 6: (E)-Methyl 4-acetoxybut-2-enoate (Compound (VI))

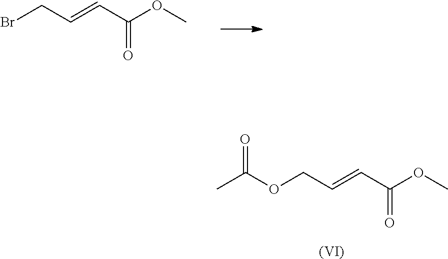

A suspension of sodium acetate (3.5 g, 42 mmol) in MeCN (30 mL) was treated with methyl 4-bromocrotonate (Aldrich) (3.33 mL, 5 g, 28 mmol) and the mixture was heated to 50° C. for 3 d. The mixture was diluted with ether and then filtered. The solid was washed with ether and the combined filtrate and washings was evaporated under reduced pressure. After evaporation the residue was partitioned between ether and water. The organic phase was washed with aqueous sodium bicarbonate, dried over MgSO₄, and evaporated under reduced pressure to give a pale orange oil. NMR indicated a mixture of product and starting material, therefore, sodium acetate (3.44 g, 42 mmol) was added to the residual oil, followed by MeCN (10 mL) and the mixture was heated to 70° C. over the weekend. The mixture was concentrated under reduced pressure and the residue was partitioned between ether and water. The organic solution was washed with water, brine, dried (MgSO₄) and filtered. The filtrate was evaporated under reduced pressure to give the title compound (3.55 g, 80%) as an orange oil: NMR δ (CDCl₃) 6.92 (dt, J 16, 5 Hz,1H), 6.01 (dt, J 16, 2 Hz, 1H), 4.72 (dd, J 5, 2 Hz, 2H), 3.73 (s, 3H), 2.10 (s, 3H).

### Intermediate 7: (E)-Methyl 4-(3-fluoro-3-(2-(5,6,7,8-tetrahydro-1,8-naphthyridin-2-yl)ethyl)pyrrolidin-1-yl)but-2-enoate (Compound (III))

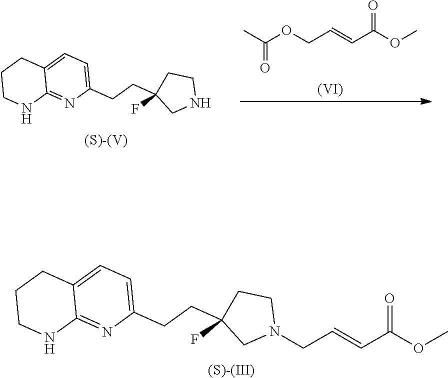

A mixture of (E)-methyl 4-acetoxybut-2-enoate (Compound (VI), for a preparation see Intermediate 6) (127 mg, 0.802 mmol), 7-(2-(3-fluoropyrrolidin-3-yl)ethyl)-1,2,3,4-tetrahydro-1,8-naphthyridine (Compound (V), for a preparation see Intermediate 5) (200 mg, 0.802 mmol) and PdCl₂(dppf)-CH₂Cl₂ adduct (65.7 mg, 0.080 mmol) in DCM (2 mL) was stirred at ambient temperature for 2 h. LCMS showed around 50% conversion and DIPEA (0.279 mL, 1.60 mmol) was added and the solution stirred for 2 h at room temperature. LCMS showed almost complete conversion to the product. The material was loaded directly onto a column and purified by chromatography (20 g amino propyl cartridge) eluting with a gradient of 0-100% EtOAc in cyclohexane over 20 min. The appropriate fractions were combined and evaporated to give the title compound (101.4 mg, 36%): LCMS (System A) RT=1.08 min, 95%, ES+ve m/z 348 (M+H)⁺. The configuration of Intermediate 8 was established as (S) and the name as (S,E)-methyl 4-(3-fluoro-3-(2-(5,6,7,8-tetrahydro-1,8-naphthyridin-2-yl)ethyl)pyrrolidin-1-yl)but-3-enoate.

### Intermediate 8: (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid and Intermediate 9: (R)-4-((S)-3-Fluoro-3-(2-(5,6,7,8-tetrahydro-1,8-naphthyridin-2-yl)ethyl)pyrrolidin-1-yl)-3-(3-(2-methoxyethoxy)phenyl)butanoic acid

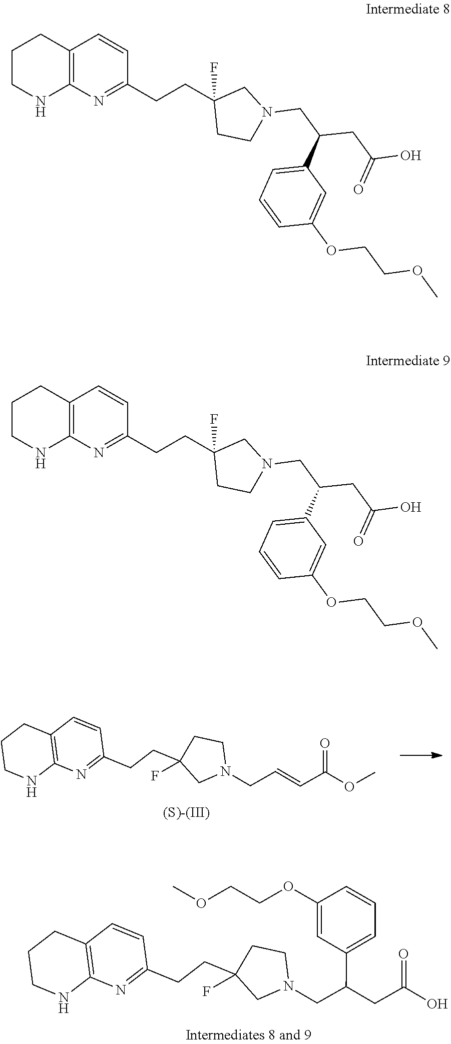 [SMILES: COCCOc1cccc([C@H](CC(=O)O)CN2CC[C@@](F)(CCc3ccc4c(n3)NCCC4)C2)c1 ; COCCOc1cccc([C@@H](CC(=O)O)CN2CC[C@@](F)(CCc3ccc4c(n3)NCCC4)C2)c1 ; COC(=O)/C=C/CN1CCC(F)(CCc2ccc3c(n2)NCCC3)C1 ; COCCOc1cccc(C(CC(=O)O)CN2CCC(F)(CCc3ccc4c(n3)NCCC4)C2)c1]

(S,E)-Methyl 4-(3-fluoro-3-(2-(5,6,7,8-tetrahydro-1,8-naphthyridin-2-yl)ethyl)pyrrolidin-1-yl)but-2-enoate (Compound (III), Intermediate 8) (101.4 mg, 0.292 mmol), 3.8M KOH (aq) (0.230 mL, 0.876 mmol) and (3-(2-methoxyethoxy)phenyl)boronic acid (compound (IV) from Enamine LLC,) (172 mg, 0.876 mmol) were dissolved in 1,4-dioxane (2 mL) and the solution was degassed. [Rh(COD)Cl]₂ (7.20 mg, 0.015 mmol) and (R)-BINAP (21.81 mg, 0.035 mmol) were suspended in 1,4-dioxane (2 mL) and degassed. The former solution of the reactants was then added to the latter catalyst solution under nitrogen. The reaction mixture was heated and stirred (50° C. 2 h). The mixture was then loaded onto an SCX cartridge (10 g) (pre-conditioned with 1CV MeOH, 1CV MeCN), washed with 10CV DMSO, 4CV MeCN, and eluted with 2M NH₃ in MeOH (4CV). The basic fraction was evaporated under reduced pressure. The residue was dried under high vacuum for 12 h to give (S)-methyl 4-(3-fluoro-3-(2-(5,6,7,8-tetrahydro-1,8-naphthyridin-2-yl)ethyl)pyrrolidin-1-yl)-3-(3-(2-methoxyethoxy)phenyl)butanoate (131.3 mg, 93%).

This methyl ester was then dissolved in THF (2 mL) and aqueous 1M LiOH (1.459 mL, 1.459 mmol) added. The solution was stirred at room temperature for 18 h. LCMS showed complete hydrolysis to the carboxylic acid and 2M HCl (0.876 mL, 1.751 mmol) was added and the solution was loaded on to a SCX cartridge (10 g) (pre-conditioned with 1CV MeOH, 1CV MeCN), washed with 4CV MeCN, and eluted with 2M NH₃ in MeOH (4CV). The basic fraction was evaporated under reduced pressure to give the crude product as a gum (127 mg, 90%). Analytical chiral HPLC RT=9.0 min, 88% and RT=13.8 min, 12% on a Chiralcel OJ-H column (4.6 mm id×25 cm) eluting with 60% EtOH (containing 0.2% isopropylamine)-heptane, flow rate=1.0 mL/min, detecting at 215 nm. The diastereoisomeric mixture was separated by preparative chiral HPLC on Chiralcel OJ-H column (3 cm×25 cm) eluting with 60% EtOH-heptane, flow rate=30 mL/min, detecting at 215 nm to give the two individual diastereoisomers of the title compound.

### Intermediate 8 (78 mg, 55%): Analytical chiral HPLC RT=9.0 min, 98.7% on a Chiralcel OJ-H column (4.6 mm id×25 cm) eluting with 60% EtOH (containing 0.2% isopropylamine)-heptane, flow rate=1.0 mL/min, detecting at 215 nm; LCMS (System D) RT=0.52 min, 100%, ES+ve m/z 486 (M+H)⁺ and (System C) RT=0.81 min, 92%, ES+ve m/z 486 (M+H)⁺ ¹H NMR (CDCl₃, 600 MHz): δ 8.45 (br s, 1H), 7.21 (t, J=7.7 Hz, 1H), 7.16 (d, J=7.2 Hz, 1H), 6.86-6.73 (m, 3H), 6.31 (d, J=7.2 Hz, 1H), 4.12 (t, J=4.4 Hz, 2H), 4.08 (br s, 1H), 3.75 (td, J=4.7, 0.8 Hz, 2H), 3.73-3.68 (m, 1H), 3.47 (br s, 2H), 3.46 (d, J=1.1 Hz, 2H), 3.42 (br t, J=5.1 Hz, 2H), 3.00-2.85 (m, 2H), 2.82-2.75 (m, 1H), 2.70-2.66 (m, 1H), 2.63-2.57 (m, 1H), 2.73-2.55 (m, 3H), 2.49 (q, J=9.1 Hz, 1H), 2.45 (dd, J=11.9, 3.7 Hz, 1H), 2.23-1.97 (m, 4H), 1.95-1.80 (m, 3H); [α]_{D}²⁰+51 (c=0.72 in ethanol).

The absolute configuration of the asymmetric centres of Intermediate 8 was determined and the compound was found to be (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid.

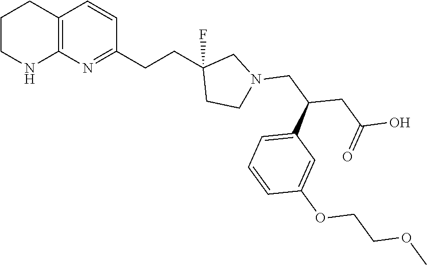 [SMILES: COCCOc1cccc([C@H](CC(=O)O)CN2CC[C@@](F)(CCc3ccc4c(n3)NCCC4)C2)c1]

Intermediate 9 (10 mg, 7%): Analytical chiral HPLC RT=12.5 min, >99.5% on a Chiralcel OJ-H column (4.6 mm id×25 cm) eluting with 60% EtOH (containing 0.2% isopropylamine)-heptane, flow rate=1.0 mL/min, detecting at 215 nm; LCMS (SystemC) RT=0.82 min, 84%, ES+ve m/z 486 (M+H)³⁰ . [α]_{D}²⁰ −28 (c=0.50 in ethanol).

The absolute configuration of the asymmetric centres of Intermediate 9 was determined and the compound was found to be of structural formula (R)-4-((S)-3-fluoro-3-(2-(5,6,7,8-tetrahydro-1,8-naphthyridin-2-ypethyppyrrolidin-1-yl)-3-(3-(2-methoxyethoxy)phenyl)butanoic acid.

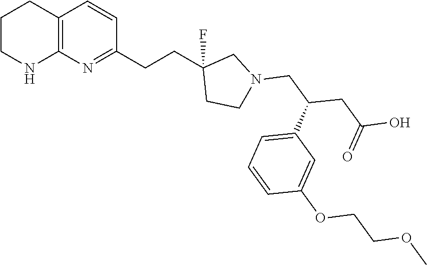 [SMILES: COCCOc1cccc([C@@H](CC(=O)O)CN2CC[C@@](F)(CCc3ccc4c(n3)NCCC4)C2)c1]

### EXAMPLE 1

### (S)-4-((S)-3-Fluoro-3-(2-(5,6,7,8-tetrahydro-1,8-naphthyridin-2-yl)ethyl)pyrrolidin-1-yl)-3-(3-(2-methoxyethoxy)phenyl)butanoic acid 1:1 citrate salt.

Citric acid (40.8 mg, 0.212 mmol) was suspended in THF (0.1 mL) and heated to 50° C. until dissolved and allowed to cool to room temperature. In a separate vial (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid (Intermediate 8) (102 mg, 0.210 mmol) was dissolved in acetonitrile (0.100 mL) and added to the citric acid solution. After approximately 10 seconds precipitation was observed. Diisopropyl ether (5 mL) was added, further precipitation occurred and the suspension was stirred for 3 h. The solid was collected by filtation and washed with diisopropyl ether (5 mL) to afford (S)-4-((S)-3-fluoro-3-(2-(5,6,7,8-tetrahydro-1,8-naphthyridin-2-yl)ethyl)pyrrolidin-1-yl)-3-(3-(2-methoxyethoxy)phenyl)butanoic acid citrate (1:1 salt) (138 mg, 0.204 mmol, 97%) as a white solid: LCMS (System C) RT=0.82 min, 100%, ES+ve m/z 486 (M+H)⁺; ¹H NMR (600 MHz, DEUTERIUM OXIDE) δ 7.54 (d, J=7.5 Hz, 1H), 7.39 (t, J=8.0 Hz, 1H), 7.03 (d, J=8.0 Hz, 1H), 6.99-6.97 (m, 1H), 6.98-6.97 (m, 1H), 6.58 (d, J=7.5 Hz, 1H), 4.22-4.20 (m, 2H), 3.83-3.81 (m, 2H), 3.79-3.70 (m, 1H), 3.70-3.65 (m, 1H), 3.67-3.61 (m, 2H), 3.64-3.60 (m, 1H), 3.55-3.47 (m, 1H), 3.49-3.45 (m, 1H), 3.47-3.43 (m, 2H), 3.44 (s, 3H), 2.86-2.83 (m, 2H), 2.86-2.81 (m, 2H), 2.80-2.75 (m, 2H), 2.74 (d, J=15.0 Hz, 2H), 2.70 (dd, J=8.0, 15.5 Hz, 1H), 2.60 (dd, J=8.0, 15.5 Hz, 1H), 2.44-2.38 (m, 1H), 2.30-2.18 (m, 1H), 2.31-2.18 (m, 2H), 1.94-1.87 (m, 2H).

### Biological Assays

### Cell Adhesion Assays

Reagents and methods utilised are as described [Ludbrook et al, Biochem. J. 2003, 369, 311 and Macdonald et al. ACS Med. Chem. Lett. 2014, 5, 1207-1212 for αᵥβ₈ assay), with the following points of clarification. The following cell lines are used, with ligands in brackets: K562-αᵥβ₃ (LAP-b₁), K562-αᵥβ₅ (Vitronectin), K562-αᵥβ₆ (LAP-b₁), K562-αᵥβ₈ (LAP-bi), A549- αᵥβ₁ (LAP- b₁). The divalent cation used to facilitate adhesion is 2 mM MgCl₂. Adhesion is quantified by cell labelling with the fluorescent dye BCECF-AM (Life Technologies), where cell suspensions at 3×10⁶ cells/mL are incubated with 0.33 uL/mL of 30 mM BCECF-AM at 37° C. for 10 minutes, then 50 μL/well are dispensed into the 96-well assay plate. At the assay conclusion cells that adhered are lysed using 50 μL/well of 0.5% Triton X-100 in H₂O to release fluorescence. Fluorescence intensity is detected using an Envision® plate reader (Perkin Elmer). For active antagonists in the assay, data is fitted to a 4 parameter logistic equation for IC₅₀ determinations.

The compound of Example 1 was tested according to the above assays and was found to be an αᵥβ₆ integrin antagonist. Those of skill in the art will recognise that in vitro binding assays and cell-based assays for functional activity are subject to experimental variability. Accordingly, it is to be understood that the values given below are exemplary only and that repeating the assay run(s) may result in somewhat different pIC₅₀ values.

The mean affinities (pIC₅₀) for Example 1 in the cell Adhesion Assays was for: αᵥβ₆ pIC₅₀=7.9; αᵥβ₃ pIC₅₀=7.2; αᵥβ₅ pIC₅₀=ND (not determined); αᵥβ₈ pIC_{50 =ND; α}ᵥβ₁ pIC₅₀=6.4.

### Chemical and Physical Stability

(S)-4-((S)-3-Fluoro-3-(2-(5,6,7,8-tetrahydro-1,8-naphthyridin-2-yl)ethyl)pyrrolidin-1-yl)-3-(3-(2-methoxyethoxy) phenyl) butanoic acid (1:1) citrate salt has been shown to have a suitable chemical stability profile (see Table 1 and Table 2) at the various conditions tested other than at 50° C., and a suitable physical stability profile (see Table 2) when the (1:1) citrate salt is protected from moisture.

The stability of the compound of the invention was determined by exposing two batches of sample (first batch—see Table 1; second batch—see Table 2) to various temperature and humidity conditions. The content of (S)-4-((S)-3-Fluoro-3-(2-(5,6,7,8-tetrahydro-1,8-naphthyridin-2-yl)ethyl)pyrrolidin-1-yl)-3-(3-(2-methoxyethoxy) phenyl) butanoic acid and impurities was measured using a high performance liquid chromatography (HPLC) analysis method, with the 5° C./amb sample as the standard for the first batch (Table 1) and the refrigerated sample as the standard for the second batch (Table 2). The impurities were determined as a percentage area relative to the(S)-4-((S)-3-Fluoro-3-(2-(5,6,7,8-tetrahydro-1,8-naphthyridin-2-yl)ethyl)pyrrolidin-1-yl)-3-(3-(2-methoxyethoxy)phenyl)butanoic acid peak in the chromatogram.

For Table 1 and Table 2 the experiments were each conducted in duplicate. The Assay values are given as a mean of the two duplicates. The Impurities values represent the results for each of the duplicate experiments.

TABLE 1
Solid State Stability
Assay (% w/w)

| | | 4 week |
|---|---|---|
| Condition/Time | 2 week | (physical state) |
| 5° C./amb | 100.1 | 99.9 (solid) |
| 30° C./65% RH E | 100.7 | 100.2 (gum) |
| 40° C./25% RH E | 99.5 | 99.1 (gum) |
| 40° C./75% RH E | 100.4 | 99.5 (gum) |
| 50° C./amb | 100.3 | 99.3 (solid) |

Impurities (% area)

| Condition/Time | 2 week | 4 week |
|---|---|---|
| 5° C./amb | 1.5, 1.4 | 1.4, 1.3 |
| 30° C./65% RH E | 1.4, 1.5 | 1.2, 1.3 |
| 40° C./25% RH E | 1.5, 1.6 | 1.5, 1.4 |
| 40° C./75% RH E | 1.4, 1.4 | 1.5, 1.5 |
| 50° C./amb | 1.6, 1.7 | 1.7, 1.9 |

E = Exposed (sample container uncapped)
RH = relative humidity
amb = ambient humidity

TABLE 2
Solid State Stability
Assay (% w/w)

| | | 4 week |
|---|---|---|
| Condition/Time | 2 week | (physical state) |
| Refrigerated | 100.0 | 99.9 (solid) |
| RT | 99.2 | 99.1 (solid) |
| 40° C. | 99.0 | 98.5 (solid) |
| 50° C. | 97.8 | 95.6 (solid but |
| | | some changes) |

Impurities (% area)

| Condition/Time | 2 week | 4 week |
|---|---|---|
| Refrigerated | 0.97, 1.0 | 1.3, 1.3 |
| RT | 1.0, 1.0 | 1.1, 1.3 |
| 40° C. | 3.5*, 1.0 | 1.2, 1.3 |
| 50° C. | 1.6, 1.4 | 2.4, 2.8 |

All stored with lid on and with desiccant
*Contained contaminant peak, only seen in one of the experiments
RT = room temperature

1-14. (canceled)

15. A compound which is (S)-4-((S)-3-fluoro-3-(2-(5,6,7,8-tetrahydro-1,8-naphthyridin-2-yl)ethyl)pyrrolidin-1-yl)-3-(3-(2-methoxyethoxy) phenyl) butanoic acid (1:1) citrate salt.

16. A pharmaceutical composition comprising the compound according to claim 15 and a pharmaceutically acceptable carrier, diluent, or excipient.

17. The pharmaceutical composition according to claim 16 wherein the pharmaceutical composition is in a form adapted for oral administration.

18. The pharmaceutical composition according to claim 17 wherein the form adapted for oral administration is a capsule.

19. A method of treating a disease or condition in a human, wherein the disease or condition is responsive to antagonism of an αᵥβ₆ receptor, the method comprising administering to the human in need thereof a therapeutically effective amount of the compound or a pharmaceutically acceptable salt thereof according to claim 15.

20. A method according to claim 19 wherein the disease or condition is a fibrotic disease.

21. A method as claimed in claim 20 wherein the fibrotic disease is idiopathic pulmonary fibrosis.
